SEQUENCE LISTING (2 sequences)

SEQ ID NO: 1 - DNA, 20 bp, Homo sapiens
  modified base (4): i
cctnccccaatccctttatt

SEQ ID NO: 2 - DNA, 20 bp, Homo sapiens
aataaagggattggggcagg

(19) United States
(12) Patent Application Publication
(10) Pub. No.: US 2006/0011474 A1
(43) Pub. Date: Jan. 19, 2006

(54) Device for detecting an analyte

(75) Inventors: Jurgen Schulein, Spardorf (DE); Christine Kugler, Spardorf (DE); Burcu Meric, Erlangen (DE); Hans Kosak, Bonn (DE); Jorg Hassmann, Erlangen (DE); Bjorn Grassl, Nurnberg (DE); Dirk Kuhlmeier, Nurnberg (DE)

Correspondence Address: KANESAKA BERNER AND PARTNERS LLP, SUITE 300, 1700 DIAGONAL RD, ALEXANDRIA, VA, 22314-2848, US

(73) Assignee: NOVEMBER AKTIENGESELLSCHAFT, Erlangen (DE)

(21) Appl. No.: 10/518,498

(22) PCT Filed: Jun. 27, 2003
(86) PCT No.: PCT/EP03/06818

§ 371 Date: Mar. 14, 2005

(30) Foreign Application Priority Data
Jun. 28, 2002 (DE) 102 29 210.8

(51) Int. Cl. G01N 27/26; G01N 33/487

(52) U.S. Cl. 204/403.01; 205/777.5; 29/592.1

(57) ABSTRACT

The invention relates to a device (17) for detecting an analyte in a liquid having a multitude of electrodes (15), which are insulated from one another while being arranged on a first side (12) of an electrically non-conductive plate (10) that is impermeable to the liquid. Said electrodes (15) have, at least in part, an analyte-specific coating or analyte-specific molecules. Electrical conductors that pass through the plate (10) can, from a second side (14) of the plate, be electrically contacted and individually discharged. The coating or the molecules has/have a specific affinity to the analytes or to a substance formed due to the presence of the analyte. In addition, the device has no leakages.

The invention relates to a device for detecting an analyte contained in a liquid and a measuring device. The analyte may be present in dissolved or suspended form. Furthermore, the invention relates to a method for producing and electrically contact-connecting the device. Moreover, the invention relates to a use of the device for detecting an analyte.

DE 197 08 529 C1 discloses a fluid sensor for liquid and gaseous organic compounds. The fluid sensor has an electrical sensor resistor that is variable in its electrical conductivity owing to penetrating fluid. The sensor resistor is applied on a nonconductive substrate. It comprises a non-conductor through which the relevant fluid can diffuse and carbon particles embedded therein. The sensor resistor can be contact-connected by means of electrodes which are contact-connected through through-holes of the substrate to contact areas on the rear side of the substrate. The contact areas produce an electrical connection between a plurality of the electrodes. The fluid sensor is suitable only for detecting organic compounds which alter the conductivity of the sensor resistor. It is not suitable for detecting other analytes.

Sosnowsky et al., (1997) Proc. Natl. Acad. Sci USA, 94, pages 1119 to 1123, disclose a silicon chip with an arrangement of electrodes for detecting a nucleic acid in a solution. Capture molecules which specifically bind analytes are immobilized on the electrodes by means of an intermediate layer. The electrodes are electrically contact-connected by lines on the surface of the chip. The lines are insulated by a silicon nitride layer. By applying a negative or positive potential to the electrodes, charged analytes may be attracted to the electrodes with the capture molecules and bind to the capture molecules. Unbound or unspecifically bound analytes can be removed again from the region of the electrodes by polarity reversal. The specifically bound analyte is detected by means of fluorescence.

Furthermore, from the company Motorola a biochip sold under the designation eSensor™ is known, in the case of which gold electrodes are arranged on the surface. The gold electrodes are laterally contact-connected on the surface of the biochip. Capture molecules are immobilized at the electrodes by means of an intermediate layer. An analyte bound to an electrode by means of the capture molecules is detected by means of reporter molecules which bind to the bound analyte and have electrochemically detectable markers. The binding of said reporter molecules is detected electrochemically.

EP 0 136 362 B1 discloses a biosensor for measuring the substrate concentration of a liquid sample. The biosensor comprises an insulating substrate plate provided with an electrode system having at least one working electrode and a counterelectrode. The electrode system is covered by a porous substrate that contains an oxidoreductase, can take up liquid and contains an enzyme capable of inducing a substrate reaction that can be detected electrochemically by means of the electrode system. The sensor furthermore has an electron acceptor. Both the oxidoreductase and the electron acceptor are soluble in the liquid sample. DE 36 87 646 T3 relates to a biosensor having an electrode system such as is known from EP 0 13.6 362 B1, the electrode system principally comprising carbon and the surface of at least the measuring electrode being covered with albumin or glucose oxidase by adsorption.

What is disadvantageous about the biosensors known from EP 0 136 362 B1 and DE 36 87 646 T3 is that the porous substrate has to be exchanged after each measurement and that the biosensor is not suitable for measuring concentrations of analytes that are not a substrate of the oxidoreductase. Furthermore, it is disadvantageous that the biosensor is not suitable for measuring many different analytes on a miniaturized substrate plate.

DE 196 21 241 A1 relates to a membrane electrode for measuring the glucose concentration in liquids. Said membrane electrode comprises a basic membrane with at least one noble metal electrode arranged on one side of the basic membrane, a proton-selective ion membrane arranged on the basic membrane and the noble metal electrode and a double membrane arranged on the ion membrane, which contains glucose oxidase in a suitable medium. The membrane electrode is suitable exclusively for measuring glucose concentrations and not for detecting other analytes in a liquid.

A biosensor chip is disclosed in WO 01/75151 A2 and DE 100 15 816 A1, on which the priority of the former is based. The sensor has electrodes embedded in an insulator layer made of insulator material. DNA probe molecules are immobilized on each electrode. The sensors are part of a silicon-based biosensor chip. Connected to the electrodes are electrode terminals at which the electrical potential that is to be applied to the electrode can be fed in. The electrode terminals are connected up to an integrated electrical circuit within the chip. What is disadvantageous in this case is that the production of the biosensor chip is too expensive to enable it to be used as only a single-use sensor chip. In the case of analytes that attack or alter the probe molecules, this may be necessary, however, for reproducible measurements.

EP 0 690 134 A1 discloses a multiple-use electrochemical solid-state sensor having an electrically nonconductive substrate, a working electrode and a semipermeable membrane covering the working electrode. The working electrode contains an electrically conductive material fixed to a part of the substrate. A first part of the conductive material is covered with an electrically insulating dielectric coating and a second part of the conductive material is covered with an active layer. The active layer comprises a catalytically effective quantity of an enzyme carried by platinized carbon powder particles distributed within the active layer. The electrochemical solid-state sensor is comparatively complex in its construction and therefore expensive to produce.

U.S. Pat. No. 5,363,690 discloses a gas detector containing an exchangeable electrochemical sensor device. The electrical contact between the exchangeable sensor device and an evaluation unit for measurement signals is produced by means of an elastomeric connector. The device is not suitable for detecting an analyte in a liquid.

WO 01/13103 A1 discloses electrodes having a surface coating made of an oxidized phenol compound, a surface-active agent being integrated into the coating. Said agent can prevent the detection of specific detergent-sensitive analytes. Therefore, the electrode can only be used for detecting specific analytes.

EP 0 402 917 A2 discloses a biosensor containing at least two spaced-apart electrical lines on an electrically nonconductive carrier. An electrically conductive organic polymerized layer made of a surface-active substance is in electrical contact with the electrical lines and covers the surface between the lines. Furthermore, a sealing coating is fitted in order to protect the electrical contacts against contact with water. A layer made of organic molecules to which complementary molecules from an aqueous medium can bind is bound to the polymerized layer made of the surface-active substance.

EP 0 987 333 A2 discloses a composition for an electrical thick-film conductor for use in electrochemical sensors, which contains conductive metal particles, graphite, a thermoplastic polymer and a surface-active substance. The compound can be used for printing working electrodes for electrochemical biosensors. Owing to the sensitivity of specific analytes with respect to surface-active substances, however, such sensors are only suitable for detecting specific analytes.

The electrodes or electrode arrangements mentioned are complex to produce. Their production requires in part lithographic techniques. Their production is too expensive to enable them to be used as only single-use electrodes or electrode arrangements. In the case of high electrode densities, it is necessary to provide the outgoing lines of the electrodes in a plurality of layers, in the so-called multilayer technique. Therefore, high electrode densities are only possible with considerable production complexity. In order to prevent contact of the electrical lines to the electrodes with a solution containing the analyte, a protective layer has to be applied to the lines. Furthermore, for specific applications, e.g. as the bottom of a microfluid chamber, it is necessary for the biochip to have a smooth surface. Therefore, a compensating layer has to be applied in order to compensate for the unevennesses caused by the lines.

It is an object of the invention to avoid the disadvantages according to the prior art. In particular, the intention is to provide a device with electrodes for detecting an analyte which is simple and thus cost-effective to produce.

This object is achieved by means of the features of claims 1, 18 to 22, 35 and 38. Expedient refinements emerge from the features of claims 2 to 17, 23 to 34, 36, 37 and 39 to 51.

The invention provides a device for detecting an analyte in a liquid having a multiplicity of electrodes that are insulated from one another and are arranged on a first side of an electrically nonconductive plate that is impermeable to the liquid, the electrodes, at least in part, having an analyte-specific coating or analyte-specific molecules and being able to be electrically contact-connected and individually conducted out from a second side of the plate by means of electrical conductors extending through the plate. The coating or the molecules is/are analyte-specific by virtue of having a specific affinity for the analyte or a substance, e.g. a decomposition product of the analyte, formed owing to the presence of the analyte. The device has not outgoing lines. The electrical conductors can be connected to the plate and the electrodes. The term “electrode” is understood in purely functional fashion. It is understood to mean the part of an electrical conductor through which electrical charge carriers can be conducted into the liquid. Consequently, the electrode may be the part of the electrical conductor which is situated on the first side of the electrically nonconductive plate. However, the electrode may also be a further electrical conductor connected to the electrical conductor extending through the plate. In this case, plate is understood to mean an arbitrary, in particular flat basic body having a first and a second side. Here and hereinafter “in part” means that both a part of an individual electrode and a portion of the electrodes present altogether may have the respective feature.

The device according to the invention is simple and thus cost-effective to produce. It is not necessary to apply a protective layer in order to prevent contact between the liquid and electrode feed lines. Furthermore, it is not necessary to apply a compensating layer in order to produce a planar surface of the plate. By virtue of the lateral outgoing lines being obviated, it is possible in a very cost-effective manner to shape the device in completely plane fashion in the region outside the electrodes. As a result, the device can readily be used as the bottom of a chamber that takes up liquid without a liquidtight seal being problematic in this case. A further advantage of the device according to the invention is that a higher electrode density than with electrodes that are conducted out laterally is possible because it is not necessary to leave space free for the lines between the electrodes. The higher electrode density can be provided without a complex multilayer technique. By means of a device according to the invention having a high electrode density and analyte-specific coatings or analyte-specific molecules having, at least in part, different specificity and electrodes that can be individually conducted out, it is possible to provide a device for simultaneous detection of many different analytes. The device according to the invention may be provided as an electrode array, in which the electrodes are in each case provided with specific molecules or coatings, for detecting different analytes or analyte combinations.

The general trend in the development of biosensor chips is toward realizing ever more complex chip structures. However, these are complex to produce and ultimately too expensive for a routine sensor technology, in particular for different analytes. Known chips produced in silicon-based fashion do not have any electrical conductors that extend through the chip such that electrodes present on one side on the chip could be conducted out from the other side. Rather, at least a part of the silicon carrier is unperforated and electrodes present are ultimately conducted out laterally. Dispensing with any outgoing line whilst at the same time enabling contact-connection from the second side of the plate permits such a simple construction of the device according to the invention that this device can be produced cost-effectively in such a way that it is suitable for single use. Measurements in which the electrodes, their analyte-specific coatings or the analyte-specific molecules are attacked can be carried out reproducibly only with a device for single use. The device according to the invention can be produced in the form of a chip for a fraction of the costs required for producing a silicon-based chip. The device may thus contribute to a breakthrough in routine sensor technology. The device according to the invention can be used in an apparatus provided for contact-connecting the device. All components which are required for conducting out and measuring a signal and are not provided by the device according to the invention are provided by the apparatus in this case. More expensive components can thus be reused.

A further advantage of the invention is that the contact-connection from the second side of the plate enables short line paths. As a result, it is possible to avoid an electrical noise caused by the comparatively long line paths in the case where the electrodes are conducted out laterally. The electrical noise reduces the sensitivity of the detection and may thereby even prevent the detection of the analyte. In an advantageous refinement, the electrical conductors are formed in one piece together with the electrodes. The electrodes and the conductors may comprise the same material. This enables good contact-connectability from the second side and very cost-effective production. It is not necessary to produce an electrical contact between the electrodes and the electrical conductors of the first side of the plate.

The coating or the analyte-specific molecules at the electrodes may in each case be different, so that different electrodes thereby differ from one another. As a result, the analyte-specific coatings or analyte-specific molecules may have a different specificity and enable an, in particular simultaneous, detection of different analytes. In this case, a detectable analyte is a member of a group that is prescribed by the specificity of the different coatings or molecules.

The coating or the analyte-specific molecules may comprise, in particular electrochemically inert, capture molecules. In this case, capture molecules are molecules to which the analyte or a substance formed owing to the presence of the analyte, e.g. a decomposition product of the analyte, binds from the liquid. The capture molecules are electrochemically inert if they do not cause a signal in the event of an electrochemical detection of the analyte. The capture molecules may be, in particular single-stranded, nucleic acids, nucleic acid analogs, ligands, haptens, peptides, proteins, sugars, lipids or ion exchangers. The capture molecules may be covalently and/or directionally bound to the electrodes. The advantage of the covalent bond is that the capture molecules cannot diffuse away from the electrodes. In the case of the very small distances between the electrodes that are possible with the device according to the invention, even capture molecules diffusing away to a small extent may lead to disruption of a detection reaction. A directional bond is to be understood to mean that the capture molecules are bound to the electrodes in each case by a specific site of the capture molecule, e.g. by one end of the molecule. It can thereby be ensured that the site of the capture molecules which is responsible for binding the analyte is not influenced by the binding of said capture molecules to the electrodes. The capture molecules, at least in part, may be bound to the electrodes by means of an, in particular electrochemically largely inert, intermediate layer. Said intermediate layer may be formed from silane. The intermediate layer is electrochemically largely inert if it does not cause a signal in the event of an electrochemical detection of the analyte.

In a preferred refinement, the coating comprises at least one semipermeable covering of the electrodes. The semipermeable coverings may in each case have a different permeability, so that the coverings of different electrodes may differ in their permeability. The coverings may be selectively permeable for molecules up to a specific size. A polymeric matrix with a molecular sieve action may be involved in this case. As a result, it is possible to permit only small molecules, arising e.g. from a specific decomposition of an analyte, to penetrate through to the electrodes, so that specifically only these molecules are detected. Such a device according to the invention can be used in a process control for tracking reactions taking place in a reactor.

The electrical conductors may be arranged in perforations of the plate which taper from the second side of the plate, in particular conically, toward the first side. In this case, the electrical conductor may be arranged only at the tapered section of the cut-out formed by the tapering form of the perforation. However, it can also project freely into the cut-out. The tapering form of the cut-out facilitates the electrical contact-connection from the second side because a conductor led in the direction of the electrode for the purpose of contact-connection is led up to the electrode even when it initially only impinges into the cut-out.

The plate may be arranged on the bottom of a microfluid chamber or form the bottom of a microfluid chamber. The device according to the invention is well suited to this owing to the possibility of the particularly planar embodiment and the associated good sealing capability.

The device may also be a chip. This is understood here to mean a small plate with electronic microstructures that does not necessarily comprise semiconductor material. In this case, the electrodes may be arranged in the form of an electrode array.

The plate may have more than 10, preferably more than 20, 40, 80, 100 or 160, particularly preferably more than 1000, especially more than 10,000 electrodes per cm². The electrodes, at least in part, may be formed from particles. The particles may be provided with analyte-specific coating or contain analyte-specific molecules. In this case, the particles may be loosely or fixedly connected among one another. A loose connection may be provided e.g. by the particles being paramagnetic and being held by magnetic force at the electrode or the electrical conductor.

Furthermore, the electrodes, at least in part, may be formed from a non-metallic conductor, in particular carbon. Carbon-containing electrodes are particularly well suited to the detection of biomolecules. The electrodes, at least in part, may be pencil, glassy carbon, carbon fiber containing, carbon paste or plastic composite electrodes, preferably polycarbonate electrodes containing elementary carbon, in particular in the form of graphite or carbon black. The carbon black may be industrial carbon black or synthetic carbon black.

The invention furthermore relates to a measuring device, comprising a device according to the invention, in which the electrodes comprise at least one reference electrode and at least one counterelectrode and also a multiplicity of working electrodes. The measuring device contains current/voltage converters, a potentiostat and a means for measuring the currents flowing through the working electrodes. The electrodes are electrically connected to the potentiostat for generating a predetermined voltage profile between the working electrodes and the reference electrode, one of the current/voltage converters being connected downstream of each of the working electrodes in order to hold all the working electrodes at the same potential. In this case, only a single potentiostat is required for generating an identical predetermined voltage profile that is applied simultaneously to all the working electrodes. By virtue of all the working electrodes being held at the same potential, it is possible, for example, for the currents flowing through the working electrodes to be measured in parallel. For this purpose, each of the working electrodes may be virtually connected to the circuit ground by means of a current follower for individual evaluation of the signals.

The invention furthermore relates to a method for producing a device according to the invention having the following steps of:
a) producing a composite of elongate electrode material that is essentially arranged parallel and insulating material surrounding the electrode material, the composite being produced by means of
   encapsulating a solid electrode material with a curing insulating material,
   introducing a solid electrode material into essentially parallel cut-outs or perforations of a solid insulating material or into a plastically deformable insulating material,
   filling pasty or liquid curing electrode material into essentially parallel cut-outs or perforations of a solid one-piece insulating material or of a stacked plate-type insulating material with congruently arranged perforations,
   connecting electrode material, having a sheathing comprising insulating material, by melting, potting or adhesively bonding the sheathing, or
   extruding a composite made of electrode material surrounded by insulating material, and
b) separating the composite essentially perpendicularly to the longitudinal direction of the electrode material by cutting, sawing or by means of a separating disk or by taking apart the stacked plate-type insulating material.

The solid electrode material may be, for example, a plurality of pencil leads which are arranged parallel and are encapsulated with epoxy resin. The plastically deformable insulating material may adapt itself to the form of the electrode material in the course of introduction and/or be adapted thereto after introduction by pressing them together. A liquidtight termination is thereby ensured. Here and herinafter “curing” of the electrode material is understood to mean that the originally liquid or pasty electrode material solidifies over time, i.e. its hardness increases. This may be effected e.g. by polymerization, by drying or by cooling of an electrode material that is pasty at a higher temperature. However, the final state of the electrode material after solidification can still be comparatively soft.

The solid one-piece insulating material may be produced by means of an injection molding method. When filling the electrode material into the stacked plate-type insulating material, the perforations are arranged such that electrode material that is filled in on one side of the stacked insulating material fills all perforations. The electrode material can be pressed into the perforations e.g. by extrusion. The method used for this purpose may be a method known from the production of pencil leads.

The sheathing can be melted by heating or chemically, e.g. by addition of a solvent that incipiently dissolves the sheathing.

When producing the composite by means of extruding the composite made from electrode material surrounded by insulating material, both the conductive electrode material and the insulating material are plastically deformable such that both materials can be extruded jointly as a composite. This enables a very cost-effective production.

Furthermore, the invention relates to a method for producing a device according to the invention having the following steps of:
a) providing an electrically nonconductive plate with perforations,
b) applying a pasty curing electrode material to a first side of the plate,
c) pressing the electrode material into the perforations, and
d) removing the electrode material present between the perforations in so far as said electrode material electrically conductively connects the electrode material present in the perforations.

The curing may be effected e.g. by polymerization, by drying or by cooling. Step ref. c may be carried out at the same time as the application in accordance with step ref. b or afterward. The method may be carried out in the manner of a screen printing method, the electrode material being applied instead of the ink.

The invention additionally relates to a method for producing a device according to the invention having the following steps of:
a) providing an electrically nonconductive plate with perforations,
b) placing an aperture mask having holes that correspond to the perforations, at least in part, or a screen printing mask having permeable areas that correspond to the perforations, at least in part, onto the first side of the plate such that the holes or the areas are congruent, at least in part, with the perforations of the plate,
c) applying a pasty curing electrode material to the aperture mask or screen printing mask,
d) pressing the electrode material into the perforations by way of the holes or permeable areas, and
e) removing the aperture mask or screen printing mask from the plate.

The method has the advantage that, through step ref. e, the removal of excess electrode material is significantly simplified and it enables a larger electrode surface because the electrodes are elevated on the first side of the plate due to the height of the aperture mask or screen printing mask. By virtue of the fact that, in the case of the same plate, different perforations are covered and left open by means of the aperture mask or screen printing mask during repeatedly effected steps ref. b to ref. e, different electrode material can be pressed into the perforations. In particular, the electrode material may have different analyte-specific molecules.

The invention moreover relates to a method for producing a device according to the invention, having the following steps of:
a) providing an electrically nonconductive plate,
b) producing perforations in the plate,
c) producing vias in the perforations for producing the electrical conductor extending through the plate and
d) applying a pasty curing electrode material to the vias on the first side of the plate.

In step ref. b the perforations may be produced by boring, in particular by means of a laser beam.

A via is an electrically conductive connection between two layers, which are formed here by the first and second sides of the electrically nonconductive plate. The via is generally used in a circuit board or an integrated circuit. Methods for producing vias are generally known. In the case of the method according to the invention, the vias are preferably produced such that they do not project beyond the plane formed by the first side of the plate. The lateral extent of the vias should be small enough that the form of the electrodes applied to the endings of the vias on the first side, preferably by the screen printing method, is not influenced thereby. Such influencing is possible because the vias often have a tubular opening in their interior. If said opening is too large, pasty or liquid electrode material applied to the via may penetrate the opening and, instead of sealing the latter, may result in the plate or the device according to the invention becoming permeable to liquid. Preferably, the vias, at their end located on the first side of the plate, have an, in particular smooth, surface that is continuous, i.e. does not have an opening.

The vias may comprise a thin copper layer, for example. Preferably, in step ref. c the vias are produced by electrodeposition in the perforations or by introducing a respective conductor into the perforations. The electrode material may be applied by means of pad printing or a method like screen printing. Both techniques are known in principle for the production of electrodes. Here they enable a particularly cost-effective and exact fabrication of the device according to the invention. In the case of pad printing, a pasty electrode material arranged in a pattern that corresponds to the desired electrode pattern is taken up by a pad. The electrode material is then applied to the electrically nonconductive plate by pressing on the pad in the form of the predetermined pattern. The electrodes produced by means of the method like screening printing are referred to as “screen printing electrodes”.

An, in particular analyte-specific, coating may be applied to the electrode material. It is also possible for analyte-specific molecules to be introduced into the electrode material. Both operations may be carried out before, after or during each of the steps mentioned. Electrode material in the sense of the invention encompasses both the material serving to produce the electrodes and the electrodes formed therefrom. Capture molecules, in particular electrochemically inert capture molecules, may be applied or introduced into the electrode material as coating or analyte-specific molecules. In each case different coatings may be applied to the electrodes or the electrode material. In each case different analyte-specific molecules may be introduced into the electrode material. The capture molecules used may be, in particular single-stranded, nucleic acids, nucleic acid analogs, ligands, haptens, peptides, proteins, sugars, lipids or ion exchangers. The capture molecules may be covalently and/or directionally bound to the electrode material or synthesized or electrochemically deposited on the electrode material. Preferably, the capture molecules, at least in part, are bound to the electrode material by means of an, in particular electrochemically largely inert, intermediate layer or are synthesized on the intermediate layer. The intermediate layer is preferably formed from silane. The electrode material may be coated with at least one semipermeable covering. This may also be effected in addition to the coating with capture molecules. The electrode material or the electrodes may in each case be coated with semipermeable coverings having different permeability. Each electrode formed from the electrode material may have a different coating.

The invention furthermore relates to a method for electrically contact-connecting a device according to the invention, a plurality of electrical conductors that can be individually conducted out being brought into contact with the second side of the plate of the device such that the conductors in this case, at least in part, contact-connect the electrodes such that the electrodes can be individually electrically conducted out. Preferably, the conductors are mounted in a manner enabling spring deflection and are brought into contact with the second side of the plate such that they effect spring deflection in this case. By way of example, a contact plate with spring pins may be used for this purpose. The electrical contact-connection may also be effected by means of an elastomeric connector, in particular a ZEBRA® elastomeric connector. Elastomeric connectors comprise alternate layers of electrically conductive and electrically nonconductive elastomer, in particular silicone elastomer. The elastomeric connectors may be formed in sheetlike fashion, the layers running perpendicular to a surface. Conductive fibers or particles, e.g. made of silver, gold or carbon, are added to the electrically conductive layer. ZEBRA® elastomeric connectors are sold by the company Fujipoly America Corporation, 900 Milik Street P.O. Box 119, Carteret, N.J. 07008, USA. The electrodes come into contact with the conductive layers by applying the ZEBRA® elastomeric connector to the second side of the plate and exerting a slight pressure on the contact area between the plate and the ZEBRA® elastomeric connector. The electrodes can be electrically conducted out through the contact-connection of the conductive layers to an electrical evaluation unit.

Furthermore, the invention relates to the use of a device according to the invention for detecting at least one analyte in a liquid, the liquid being brought into contact with electrodes on the first side of the plate of the device and the electrodes being electrically contact-connected from the second side of said plate. In this case, the liquid is preferably brought into contact with the electrodes under conditions under which the analyte or a substance, e.g. a decomposition product of the analyte, formed owing to the presence of the analyte binds to capture molecules present at the electrodes. The detection of the analyte bound to the capture molecules or of the substance may be effected electrically, e.g. by conductivity measurement, electrochemically, optically, photoelectrically, enzymatically, by means or electroluminescence or by means of chemiluminescence. The detection may also be effected by means of a combination of the detection methods mentioned. In the case of electrochemical detection, it is advantageous if a direct contact between the analyte or the substance and the electrode is made possible. In the case of optical detection, it is possible to measure an optical signal, such as e.g. fluorescence, at the electrodes. The analyte or the substance is identified in this case for example by identifying by optical detection that electrode to which a fluorescent analyte or a fluorescent substance is specifically bound by means of the capture molecules. By virtue of the fact that the electrode can be assigned to a specific capture molecule, it is possible to identify the analyte or the substance. The electrodes serve for electrical attraction and/or repulsion of charged analytes or substances in the case of this detection method. By applying a corresponding potential to an electrode, the charged analytes or the charged substances can be electrically transported into the region of the capture molecules. Through an increased concentration of the analytes or substances in the region of the capture molecules, it is possible to accelerate the binding of the analytes or substances thereto. Unbound or weakly and unspecifically bound analytes or substances may be removed from the electrode by applying a repulsive potential thereto. In this case, it is advantageous if the capture molecules are immobilized at the electrodes by means of an analyte- or substance-impermeable intermediate layer. This prevents the analyte or the substance from being electrochemically converted in the event of direct contact with the electrode. This enables the application of high potentials for rapid transport of the analytes or the substances to the capture molecules.

The electrodes may be coated with a semipermeable covering. This enables the selective detection of only the analytes, decomposition products of the analytes or the substances which penetrate through the covering. The detection may be effected electrically, electrochemically, optically, photoelectrically, enzymatically, by means of electroluminescence or by means of chemiluminescence. It may also be effected by means of a combination of these detection methods. Preferably, the electrodes are in each case coated with semipermeable coverings having different permeability.

The analyte may be a biomolecule, in particular a nucleic acid, a protein, an antigen, a sugar, a lipid, a cell or a virus. It may have a labelling substance. The labelling substance may be e.g. an enzyme or a redox-active label. In the use of the device, a redox reaction or a catalytic evolution of hydrogen may be detected electrochemically. The electrochemical detection may be effected e.g. by means of differential pulse voltammetry (DPV), chronopotentiometric stripping analysis (CPSA) or detection of a change in resistance or impedance.

The electrochemical detection may comprise the following steps of:
a) providing a device according to the invention, the device having at least one counterelectrode and a reference electrode and also a multiplicity of working electrodes,
b) bringing the liquid into contact with the working, counter- and reference electrodes,
c) simultaneously applying a predetermined voltage profile between the working electrodes and the reference electrode, and
d) measuring the currents flowing through the working electrodes, all the working electrodes being held at the same potential during measurement.

A potential interval in which essentially only the analyte or the substance causes a signal is preferably chosen for measurement for the electrochemical detection.

Preferably, the, in particular carbon containing, electrodes are treated with a detergent prior to the detection of the analyte. This may be effected before or while the liquid containing the analyte is in contact with the electrodes. The treatment with detergent may replace an electrochemical conditioning. It is simpler, faster and more cost-effective than an electrochemical conditioning. The electrodes may be stored in a detergent-containing liquid and e.g. be sold in said liquid. Preferably, the detergent is an ionic detergent. The detergent is expediently present in a concentration of 0.1% to 10%. Preferably, the detergent has a critical micellar concentration of less than 10 mmol/l, in particular less than 5 mmol/l, preferably less than 3 mmol/l, in water. The detergent may be sodium dodecyl sulfate.

Exemplary embodiments of the invention are explained in more detail with reference to the drawing, in which:

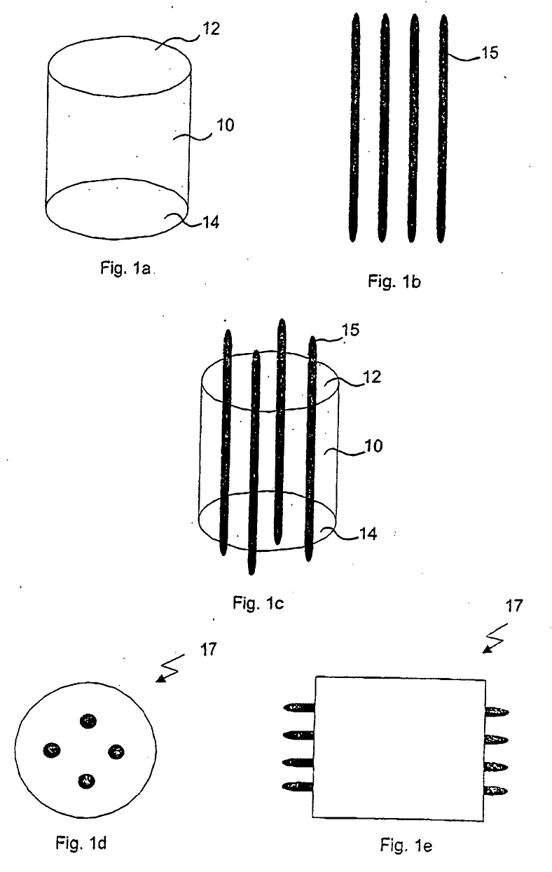

FIGS. 1a-e show a diagrammatic illustration of a method for producing the device according to the invention,

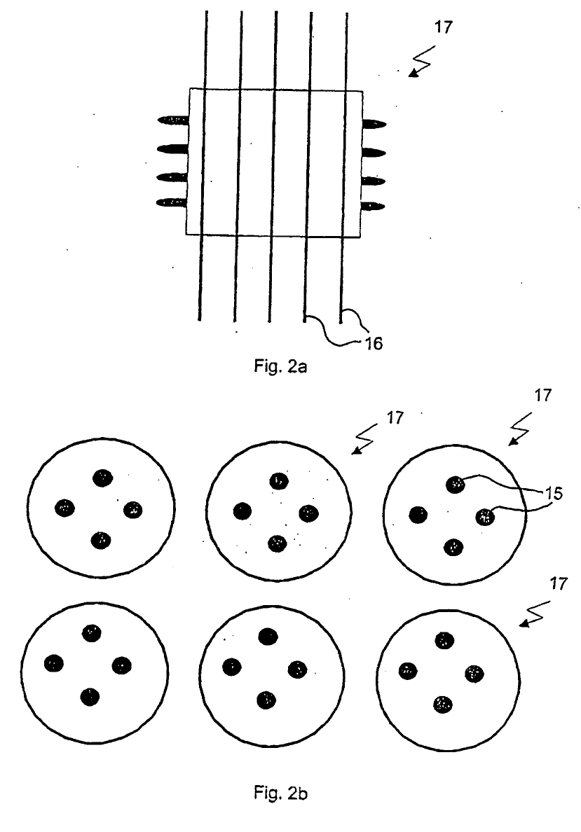

FIGS. 2a-b show a diagrammatic illustration of a method for producing a device according to the invention by means of severing a composite made of electrode material and insulating material,

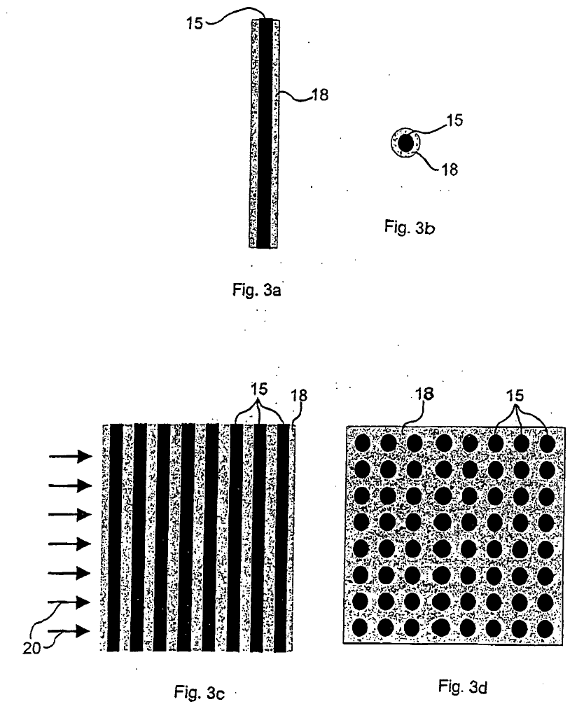

FIGS. 3a-d show a diagrammatic illustration of a method for producing a composite made of elongate electrode material arranged parallel and insulating material,

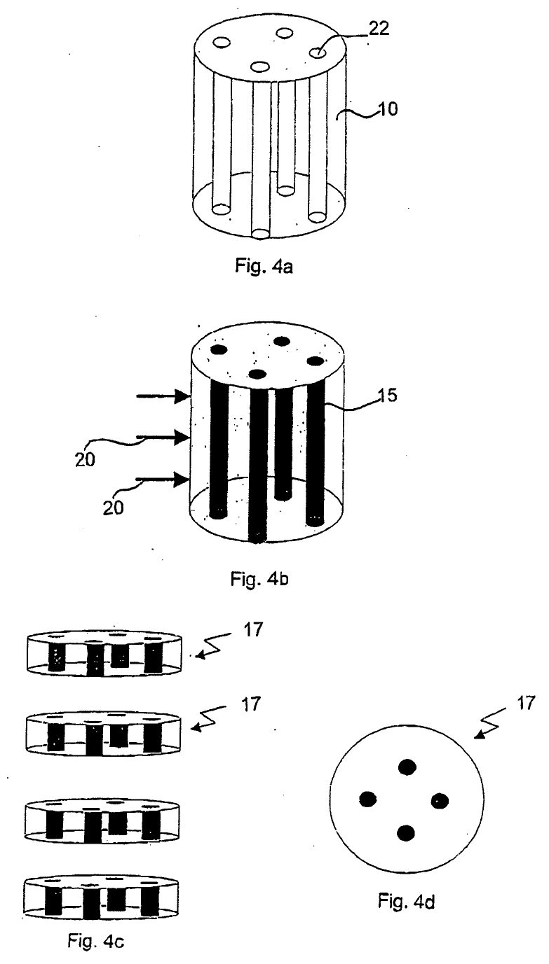

FIGS. 4a-d show a diagrammatic illustration of a method for producing a detection device according to the invention by means of extrusion and severing a composite produced thereby,

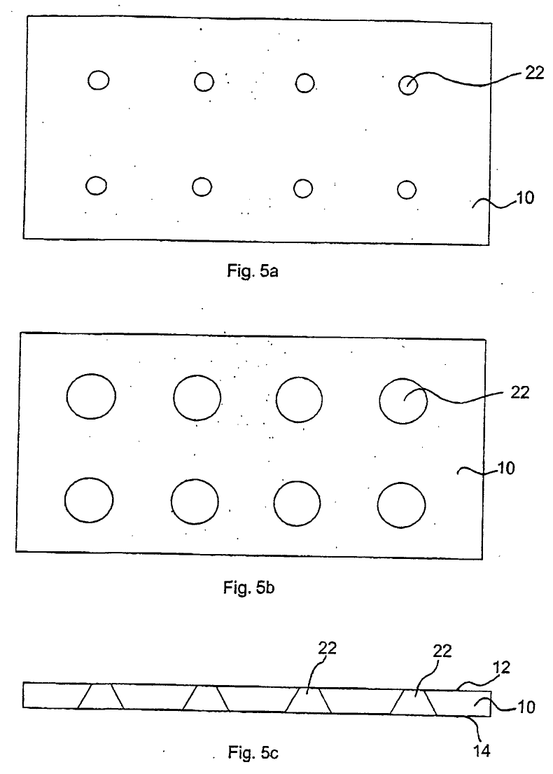

FIGS. 5a-c show a base plate for producing a detection device,

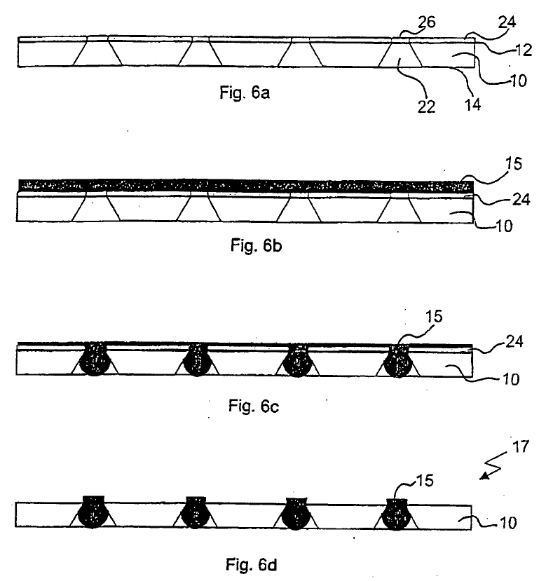

FIGS. 6a-d show a diagrammatic illustration of a method like screen printing for producing a device according to the invention,

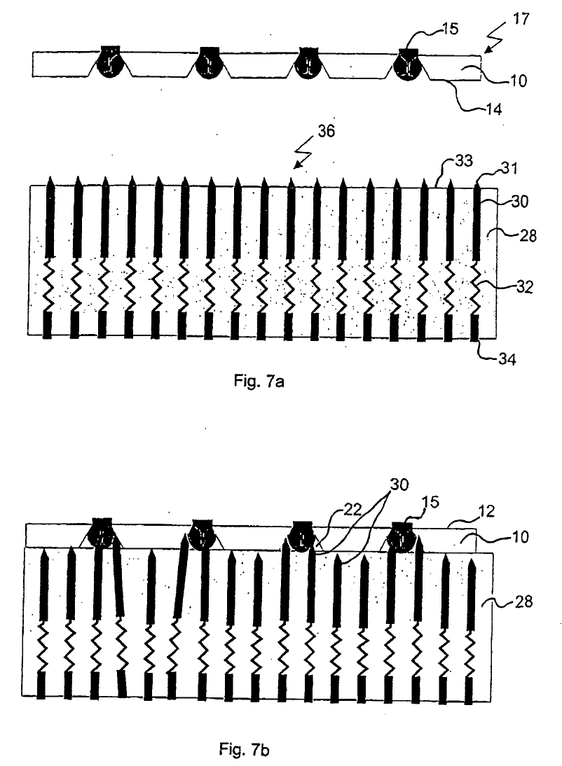

FIGS. 7a-b show a diagrammatic illustration of a method and a device for electrically contact-connecting the detection device according to the invention,

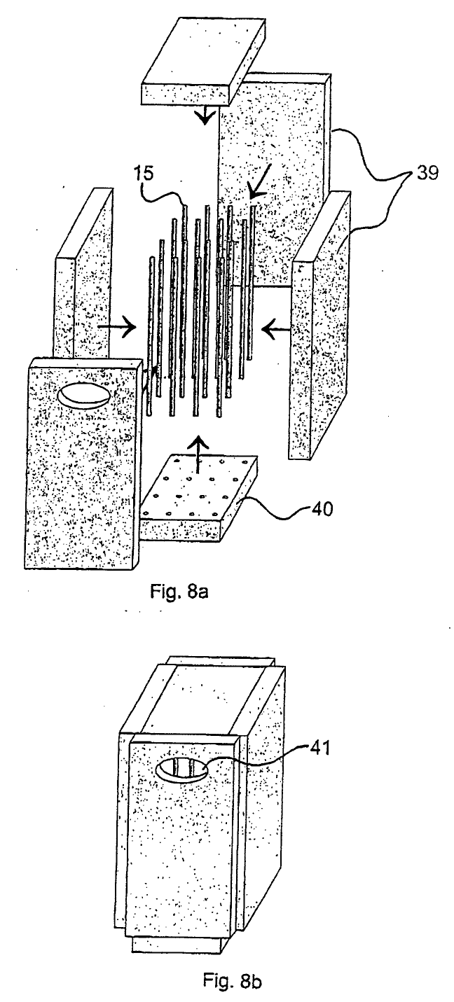

FIGS. 8a-b show a diagrammatic illustration of a method for producing a chip having 4×4 electrodes,

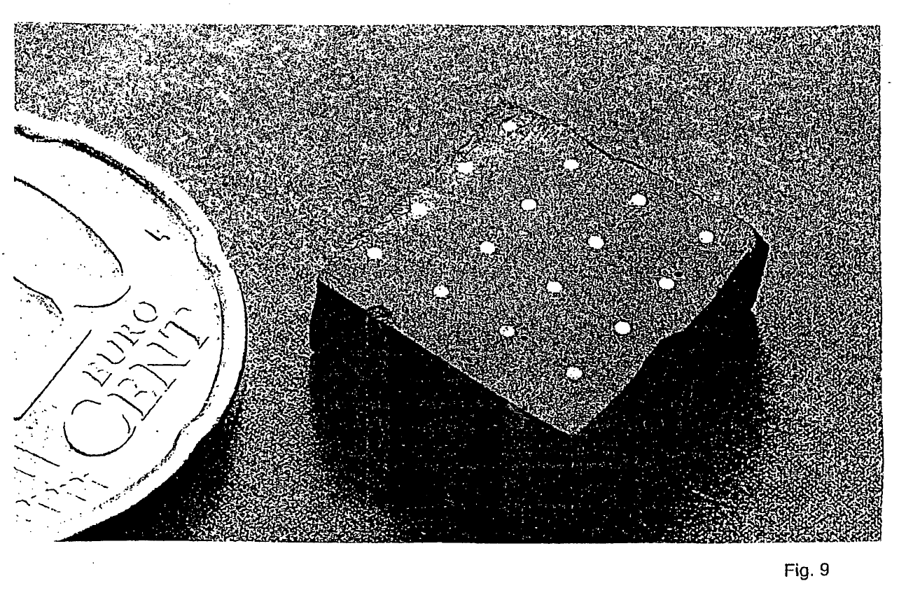

FIG. 9 shows a representation of the chip,

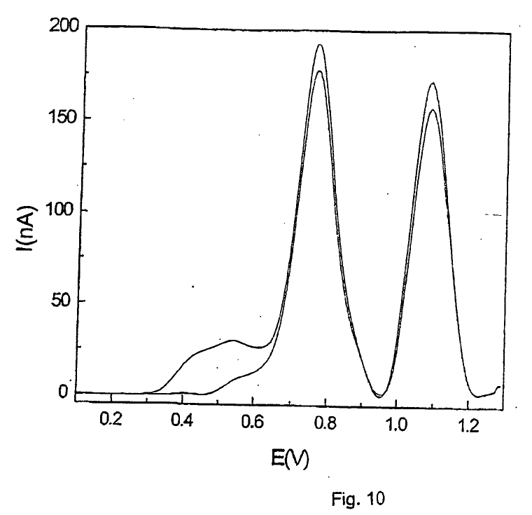

FIG. 10 shows the result of two DPV measurements of herring sperm DNA carried out in parallel using the chip, and

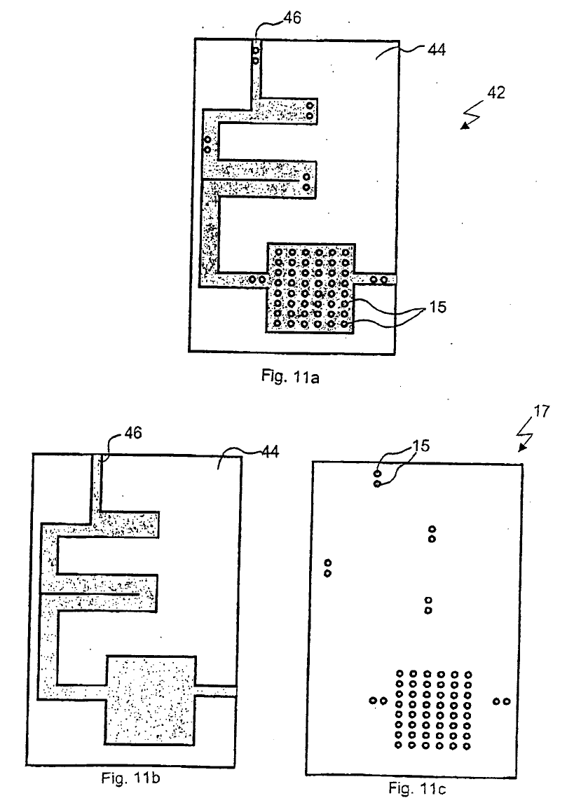

FIGS. 11a-c show a diagrammatic illustration of a microfluid chamber with the detection device according to the invention.

FIG. 1a shows a plastically deformable electrically insulating basic body 10 having a first side 12 and a second side 14. FIG. 1b shows four electrodes 15 formed from pencil leads. FIG. 1c illustrates the basic body 10 with electrodes 15 introduced therein by mechanical pressure. In this case, the electrodes are introduced in such a way that each electrode projects on the first side 12 and the second side 14. After the introduction of the electrodes 15, the basic body 10 can be cured. FIG. 1d shows the resulting detection device 17 in plan view, and FIG. 1e shows the device in side view. As illustrated in FIG. 2a, the device 17 can be multiply severed perpendicularly along the lines 16 and thereby be split into the disk-type devices according to the invention illustrated in FIG. 2b. In this case, each of the electrodes 15 is in contact with the respective top side and underside of the disks.

An electrode 15 with a sheathing 18 comprising insulating material is illustrated in cross section in FIG. 3a and in plan view in FIG. 3b. FIGS. 3c and 3d show a composite of such electrodes in cross section and in plan view, said composite resulting from connection of the sheathings 18. The arrows 20 indicate positions at which the composite can be severed in order to produce disk-type devices 17 according to the invention therefrom.

FIG. 4a shows an electrically insulating basic body 10 with four parallel first perforations 22. The basic body 10 may for example comprise a plastic and be produced by an injection molding method. A composition comprising an electrically conductive electrode material 15 may be pressed into the first perforations 22 of the basic body 10. This may be effected for example by means of an extrusion method as is customarily used for the production of pencil leads. The electrode material 15 may be a material for producing pencil leads. The basic body 10 may be severed, also before the electrode material 15 has actually cured, at the locations indicated by the arrows 20 perpendicular to the first perforations 22 filled with electrode material 15. This results in the disk-type devices 17 according to the invention which are illustrated in perspective view in FIG. 4c and in plan view in FIG. 4d. As an alternative to the mechanical severing of the composite made of electrode material 15 and basic body 10, a stack of disk-type basic bodies 10 with first perforations may be stacked one above the other such that the first perforations 22 are congruent. When the electrode material 15 is filled in at one end of the stack, then all of the first perforations 22 of the disk-type basic bodies 10 are filled. The stack can then be taken apart still before the electrode material has cured.

FIG. 5c shows a plate-type basic body 10 having a first side 12 and a second side 14 in cross section. FIG. 5b shows this basic body 10 in plan view from the second side 14 and FIG. 5a shows it in plan view from the first side 12. The basic body 10 has conical perforations 22 widening from the first side 12 to the second side 14. In FIG. 6a, the plate-type basic body 10 is covered with an aperture mask 24 on the first side 12, said aperture mask having holes 26 that are congruent with the perforations 22 on the first side 12. FIG. 6b shows electrically conductive pasty electrode material 15 applied to the aperture mask 24. FIG. 6c shows the electrode material 15 after it has been pressed into the holes 26 and the perforations 22 in a method like screen printing. FIG. 6d illustrates the device 17 according to the invention after removal of the aperture mask 24.

FIGS. 7a and 7b show a device for electrically contact-connecting 36 a detection device 17. In this case, the electrical contact-connecting device 36 comprises an elastic matrix 28 made of an electrically insulating material. Electrically conductive pins 30 are arranged parallel in said matrix 28 and are electrically connected to contacts 34 on the underside of the matrix. The pins are pressed out from the elastic matrix by a spring 32. The pins 30 preferably taper to a point at the side provided for contact-connection. The contact-connection—illustrated in FIG. 7b—of the detection device 17 by the electrical contact-connecting device 36 is effected by pressing the two devices 17, 36 against one another. In this case, the pins 30 come into contact with the electrodes 15. The elastic matrix 28 is compressed in this case. As a result, the pins 30 can penetrate the perforations 22 of the detection device 17, which taper toward the first side 12, and make contact with the electrodes 15 in this case. An enlarged contact area between the pins 30 and the electrodes 15 is provided by virtue of the form of the pins 30 tapering to a point, the tapering perforations 22 and the form of the electrodes 15.

An arrangement of claddings 39 and an electrode mount 40 for encapsulating the electrodes 15 with an insulating material, such as, for example, epoxy resin, is diagrammatically illustrated before assembly in FIG. 8a and after assembly in FIG. 8b. One of the claddings 39 has an opening 41 for filling in the insulating material. The composite made of electrodes and insulating material which results from the polymerization of the insulating material can be severed, thereby producing disk-type detection devices 17 as chips having 4×4 electrodes. Such a device 17 is shown in FIG. 9. In this case, pencil leads serve as electrode material. The electrodes of one of the chips were treated or conditioned electrochemically for 1 min with 1.2 V in 0.1 M sodium acetate buffer, pH 4.6. The electrodes of another of the chips were treated for 1 min with 10% SDS. For the purpose of silanizing the electrodes, the chips were incubated for 1 h at room temperature with slight shaking in a solution comprising 1% (v/v) 3-(glycidyloxypropyl)-trimethoxysilane (Fluka), 1% (v/v) deionized water (Millipore) and 98% (v/v) ethanol (Merck). They were subsequently dried for 30 min at 80° C.

The oligonucleotide TNF2 with the sequence 5′ cct icc cca atc cct tta tt 3′-aminolink (SEQ ID NO: 1-aminolink), where i represents an inosine moiety, was coupled as capture molecule to the silanized electrodes. The oligonucleotide is a sequence comprising the c-DNA of the human tumor necrosis factor a gene, said sequence being provided with an aminolink. For coupling purposes, in each case one drop of a solution containing 150 pmol/ml of oligonucleotide in 0.1 M Na₂CO₃, pH 9.5, was placed onto each of the electrodes of the chips. The chips were then incubated for one hour at room temperature in a humid chamber. In this case, the free amino groups of the oligonucleotides form a covalent bond with the silane. In order to separate oligonucleotides that had not formed a covalent bond, the chips were incubated for one hour in 2 ml 10% SDS at room temperature. In order to saturate binding sites still present, the chips were incubated for one hour at room temperature in 1% bovine serum albumin (BSA) or ethanolamine in phosphate buffered saline (PBS).

In order to investigate the influence of an electrode treatment on the sensitivity and reproducibility of the electrochemical nucleic acid detection, the chips were incubated in a solution of 10 nmol/ml of the complementary nucleic acid TNF2k (SEQ ID NO: 2) in detergent-containing hybridization buffer (Roche) and the bound nucleic acid TNF2k was determined by means of DPV. In each case ten measurements were carried out with the electrodes treated electrochemically or with detergent. The detergent treatment results in an increase in sensitivity of more than 10% compared with the electrochemical treatment. Furthermore, the reproducibility of the measurements was improved with detergent-treated electrodes. The standard deviation of the measurements of detergent-treated electrodes was a factor of 3 lower than in the case of an electrochemical treatment.

FIG. 10 shows two voltammograms that were determined by means of DPV measurements of herring sperm DNA carried out in parallel using the device 17 shown in FIG. 9. For this purpose, the electrode material of the device 17 was connected to an electronic evaluation unit by means of spring contact pins from the second side of said electrode material. One of the electrodes was connected as reference electrode. 100 μl of a 2 μg/μl herring sperm DNA solution in TE buffer (10 nM TrisCl, 1 mM EDTA, pH 8) were applied to the first side of the device and incubated for 10 min. The DNA was detected in parallel at a plurality of electrodes by means of DPV on the basis of the oxidation of guanine and adenine. Significant guanine and adenine oxidation peaks that are congruent in their position were measured in this case.

FIG. 11a diagrammatically shows a plan view of an assembled microfluid chamber 42 with a multiplicity of electrodes 15 and the cut-out 46 for the passage of liquid. FIG. 11b shows a plan view of the upper part 44 of the microfluid chamber 42 and FIG. 11c shows a plan view of the lower part of said chamber formed by the device 17 according to the invention.

1. A device (17) for detecting a multiplicity of different analytes in a liquid having a multiplicity of electrodes (15) that are insulated from one another and are arranged on a first side (12) of an electrically nonconductive plate (10) that is impermeable to the liquid, the plate being formed as a flat basic body having a first and a second side, the electrodes (15), at least in part, having an analyte-specific coating or analyte-specific molecules having, at least in part, different specificity and being able to be electrically contact-connected and individually conducted out from a second side (14) of the plate by means of electrical conductors extending through the plate (10), the coating or the molecules being analyte-specific by virtue of having a specific affinity for the analyte or a substance formed owing to the presence of the analyte, and the device having no outgoing lines, the plate (10) being a chip, the electrodes being arranged in the form of an electrode array.

2. The device (17) as claimed in claim 1, the electrical conductors being formed in one piece together with the electrodes (15).

3. The device (17) as claimed in claim 1, the coating or the analyte-specific molecules at the electrodes (15) in each case being different.

4. The device (17) as claimed in claim 1, the coating or the analyte-specific molecules comprising, in particular electrochemically inert, capture molecules.

5. The device (17) as claimed in claim 4, the capture molecules being, in particular single-stranded, nucleic acids, nucleic acid analogs, ligands, haptens, peptides, proteins, sugars, lipids or ion exchangers.

6. The device (17) as claimed in claim 4, the capture molecules being covalently and/or directionally bound to the electrodes (15).

7. The device (17) as claimed in claim 4, the capture molecules, at least in part, being bound to the electrodes (15) by means of an, in particular electrochemically largely inert, intermediate layer.

8. The device (17) as claimed in claim 7, the intermediate layer being formed from silane.

9. The device (17) as claimed in claim 1, the coating comprising at least one semipermeable covering of the electrodes (15).

10. The device (17) as claimed in claim 9, the semipermeable coverings in each case having a different permeability.

11. The device (17) as claimed in claim 1, the electrical conductors being arranged in perforations (22) of the plate (10) which taper from the second side (14) of the plate (10), in particular conically, toward the first side (12).

12. The device (17) as claimed in claim 1, the plate (10) being arranged on the bottom of a microfluid chamber (42) or forming the bottom of a microfluid chamber.

13. The device (17) as claimed in claim 1, the plate (10) having more than 10, preferably more than 20, 40, 80, 100 or 160, particularly preferably more than 1000, especially more than 10,000 electrodes per cm².

14. The device (17) as claimed in claim 1, the electrodes (15), at least in part, being formed from particles.

15. The device (17) as claimed in claim 1, the electrodes (15), at least in part, being formed from a non-metallic conductor, in particular carbon.

16. The device (17) as claimed in claim 15, the electrodes (15), at least in part, being pencil, glassy carbon, carbon fiber containing, carbon paste or plastic composite electrodes, preferably polycarbonate electrodes containing elementary carbon, in particular in the form of graphite or carbon black.

17. A measuring device, comprising a device (17) as claimed in claim 1, in which the electrodes (15) comprise at least one reference electrode and at least one counterelectrode and also a multiplicity of working electrodes, the measuring device containing current/voltage converters, a potentiostat and a means for measuring the currents flowing through the working electrodes, and the electrodes (15) being electrically connected to the potentiostat for generating a predetermined voltage profile between the working electrodes and the reference electrode, one of the current/voltage converters being connected downstream of each of the working electrodes in order to hold all the working electrodes at the same potential.

18. A method for producing a device (17) as claimed in claim 1 having the following steps of:
a) producing a composite of elongate electrode material (15) that is essentially arranged parallel and insulating material surrounding the electrode material (15), the composite being produced by means of
encapsulating a solid electrode material (15) with a curing insulating material,
introducing a solid electrode material (15) into essentially parallel cut-outs or perforations (22) of a solid insulating material or into a plastically deformable insulating material,
filling pasty or liquid curing electrode material (15) into essentially parallel cut-outs or perforations (22) of a solid one-piece insulating material or of a stacked plate-type insulating material with congruently arranged perforations (22),
connecting electrode material (15), having a sheathing (18) comprising insulating material, by melting, potting or adhesively bonding the sheathing (18), or
extruding a composite made of electrode material (15) surrounded by insulating material (18), and
b) separating the composite essentially perpendicularly to the longitudinal direction of the electrode material (15) by cutting, sawing or by means of a separating disk or by taking apart the stacked plate-type insulating material.

19. A method for producing a device (17) as claimed in claim 1 having the following steps of:
a) providing an electrically nonconductive plate (10) with perforations (22),
b) applying a pasty curing electrode material (15) to a first side (12) of the plate (10),
c) pressing the electrode material (15) into the perforations (22), and
d) removing the electrode material (15) present between the perforations (22) in so far as said electrode material (15) electrically conductively connects the electrode material (15) present in the perforations.

20. A method for producing a device (17) as claimed in claim 1 having the following steps of:
a) providing an electrically nonconductive plate (10) with perforations (22),
b) placing an aperture mask (24) having holes (26) that correspond to the perforations (22), at least in part, or a screen printing mask having permeable areas that correspond to the perforations, at least in part, onto the first side (12) of the plate (10) such that the holes (26) or the areas are congruent, at least in part, with the perforations (22) of the plate (10),
c) applying a pasty curing electrode material (15) to the aperture mask (24) or screen printing mask,
d) pressing the electrode material (15) into the perforations (22) by way of the holes or permeable areas, and
e) removing the aperture mask (24) or screen printing mask from the plate (10).

21. The method as claimed in claim 18, an analyte-specific coating being applied to the electrode material (15) or analyte-specific molecules being introduced into the electrode material (15).

22. The method as claimed in claim 21, capture molecules, in particular electrochemically inert capture molecules, being applied or introduced as coating or analyte-specific molecules.

23. The method as claimed in claim 21, in each case different coatings being applied to the electrode material (15) or in each case different analyte-specific molecules being introduced into the electrode material (15).

24. The method as claimed in claim 21, the capture molecules used being, in particular single-stranded, nucleic acids, nucleic acid analogs, ligands, haptens, peptides, proteins, sugars, lipids or ion exchangers.

25. The method as claimed in claim 21, the capture molecules being covalently and/or directionally bound to the electrode material (15) or being synthesized or electrochemically deposited on the electrode material (15).

26. The method as claimed in claim 21, the capture molecules, at least in part, being bound to the electrode material (15) by means of an, in particular electrochemically largely inert, intermediate layer or being synthesized on the intermediate layer.

27. The method as claimed in claim 26, the intermediate layer being formed from silane.

28. The method as claimed in claim 18, the electrode material (15) being coated with at least one semipermeable covering.

29. The method as claimed in claim 28, the electrode material (15) in each case being coated with semipermeable coverings having different permeability.

30. The use of a device (17) as claimed in claim 1 for detecting at least one analyte in a liquid, the liquid being brought into contact with electrodes (15) on the first side (12) of the plate (10) of the device (17) and the electrodes (15) being electrically contact-connected from the second side (14) of said plate.

31. The use as claimed in claim 30, the liquid being brought into contact with the electrodes (15) under conditions under which the analyte or a substance formed owing to the presence of the analyte binds to capture molecules present at the electrodes (15) and the analyte bound to the capture molecules or the substance bound thereto is detected electrically, electrochemically, optically, photoelectrically, enzymatically, by means of electroluminescence or by means of chemiluminescence or by means of a combination thereof.

32. The use as claimed in claim 30, at least one electrode (15) being coated with a semipermeable covering and selectively only such analytes, decomposition products of analytes or substances which penetrate the covering being detected electrically, electrochemically, optically, photoelectrically, enzymatically, by means of electroluminescence or by means of chemiluminescence or by means of a combination thereof.

33. The use as claimed in claim 30, the analyte being a biomolecule, in particular a nucleic acid, a protein, an antigen, a sugar, a lipid, a cell or a virus.

34. The use as claimed in claim 30, the analyte having a labelling substance.

35. The use as claimed in claim 30, a redox reaction or a catalytic evolution of hydrogen being detected electrochemically.

36. The use as claimed in claim 30, the electrochemical detection being effected by means of differential pulse voltammetry (DPV), chronopotentiometric stripping analysis (CPSA) or detection of a change in resistance or impedance.

37. The use as claimed in claim 30, the electrochemical detection comprising the following steps of:
a) providing a device (17) as claimed in one of claims 1 to 16, the device (17) having at least one counterelectrode and a reference electrode and also a multiplicity of working electrodes,
b) bringing the liquid into contact with the working, counter- and reference electrodes,
c) simultaneously applying a predetermined voltage profile between the working electrodes and the reference electrode, and
d) measuring the currents flowing through the working electrodes, all the working electrodes being held at the same potential during measurement.

38. The use as claimed in claim 30, a potential interval in which essentially only the analyte or the substance causes a signal being chosen for measurement for the electrochemical detection.
